(19) United States
(12) Patent Application Publication
(10) Pub. No.: US 2019/0082964 A1
(43) Pub. Date: Mar. 21, 2019

(54) Massively Multi-Frequency Ultrasound-Encoded Tomography

(71) Applicant: The Charles Stark Draper Laboratory, Inc., Cambridge, MA (US)

(72) Inventors: Steven J. Byrnes, Watertown, MA (US); Joseph Hollmann, Watertown, MA (US)

(21) Appl. No.: 16/133,948

(22) Filed: Sep. 18, 2018

Related U.S. Application Data
(60) Provisional application No. 62/653,646, filed on Apr. 6, 2018.
(60) Provisional application No. 62/621,100, filed on Jan. 24, 2018.
(60) Provisional application No. 62/582,391, filed on Nov. 7, 2017.
(60) Provisional application No. 62/559,779, filed on Sep. 18, 2017.

(51) Int. Cl. A61B 5/00; A61B 8/15; A61B 8/00

(52) U.S. Cl. CPC A61B 5/0073; A61B 8/15; G01N 21/1717; A61B 5/0097; A61B 8/4494

(57) ABSTRACT

A system is described for multi-frequency ultrasonically-encoded optical tomography of target tissue. A light source generates light input signals to the target tissue. An ultrasound transducer array has ultrasound transducers each generating a different time-dependent waveform to form a plurality of ultrasound input signals to an imaging volume within the target tissue. An optical sensor senses scattered light signals from the imaging volume, wherein the scattered light signals include light input signals modulated by acousto-optic interactions with the ultrasound input signals. Spectral analysis of the scattered light signals is performed to create a three-dimensional image map representing biomarker characteristics of the target tissue.

This application claims priority from U.S. Provisional Patent Application 62/653,646, filed Apr. 6, 2018, and U.S. Provisional Patent Application 62/621,100, filed Jan. 24, 2018, and U.S. Provisional Patent Application 62/582,391, filed Nov. 7, 2017, and U.S. Provisional Patent Application 62/559,779, filed Sep. 18, 2017, all of which are incorporated herein by reference in their entireties.

### BACKGROUND ART

A non-invasive three-dimensional optical video of patient tissue such as the brain using multiple wavelengths could reveal useful information including real-time spectroscopic information of the imaging volume, which can be used for highly-specific quantitative maps of many different bio-markers in parallel. This can represent information about tissue parameters such as blood oxygenation, glucose, clots, swelling, and neuron firing; see for example, “In Vivo Observations of Rapid Scattered Light Changes Associated with Neurophysiological Activity”, Rector et al. from book: In Vivo Optical Imaging of Brain Function, 2009, which is incorporated herein by reference in its entirety. This could lead to new diagnostic approaches for many medical conditions such as traumatic brain injury and tumors, and could also provide maps of brain activation patterns, with implications for psychiatric diagnostics, communication systems for paraplegics and others, control of prosthetics, and brain-machine interfaces more generally.

FIG. 1 illustrates the principle of conventional ultrasound-modulated optical tomography (see for example, “Photorefractive detection of tagged photons in ultrasound modulated optical tomography of thick biological tissues”, Ramaz et al., Optics Express 12, 5469 (2004), which is incorporated herein by reference in its entirety). Target tissue 102 such as the brain of a patient can be considered as a medium that is transparent to ultrasound, but highly scattering to light. A light source 101, an ultrasound transducer phased array 103, and an optical sensor 105 are all placed on the target tissue 102 and operated by an optical tomography processor 106 that includes at least one hardware processor and which may be coupled to data storage memory (not shown) that is configured for storing optical tomography software and other system information and signals. The tomography processor 106 is configured to execute the optical tomography software including instructions to operate the ultrasound transducers in the ultrasound transducer array 103 to focus ultrasound waves (e.g. at 5 MHz) to an imaging volume 104, which is a particular small region in three-dimensional space in the target tissue 102 (which also can be thought of and referred to as a “voxel”). The tomography processor 106 also operates the light source 101 to provide one or more light input signals to the target tissue 102. The light input signals scatter randomly in all directions, tracing complicated paths through the target tissue 102. However, some small fraction of the light signals travel from the light source 101, through the imaging volume 104, and out to the optical sensor 105. This scattered light is modulated in intensity and/or phase at 5 MHz, effectively creating optical sidebands shifted by ±5 MHz from the optical frequency. The tomography processor 106 detects these sidebands through any of several methods—most simply digitizing the received intensity and calculating the component that oscillates at 5 MHz, but alternatively using more sophisticated detection methods such as discussed as in Ramaz et al. (above). The intensity and phase of the scattered light sidebands indicates the properties of that imaging volume 104, including its light intensity, acousto-optic coefficient, etc. After measuring one imaging volume 104, the tomography processor 106 can change the ultrasound phase pattern delivered by the ultrasound transducer array 103 to measure another imaging volume, and so on.

In certain spectral windows, particularly including red and near infrared (NIR), light from non-invasive external light sources can penetrate through the skin and skull into the target tissue (e.g., the brain) sufficiently to get meaningful data out. Unfortunately, red and NIR light undergoes multiple scattering which obfuscates the spatial structure of the target tissue, thus making it very challenging to get a high-resolution spatial map. There is currently no good solution to this problem.

### SUMMARY

Embodiments of the present invention are directed to computer-implemented arrangements for multi-frequency ultrasonically-encoded optical tomography of target tissue such as a brain of a patient. A light source is configured for generating light input signals to the target tissue. An ultrasound transducer array is configured for placement on the outer surface of the target tissue and has multiple ultrasound transducers each generating a different time-dependent waveform to form a plurality of ultrasound input signals to an imaging volume within the target tissue. An optical sensor is configured for sensing scattered light signals from the imaging volume, wherein the scattered light signals include light input signals modulated by acousto-optic interactions with the ultrasound input signals. Data storage memory is configured for storing optical tomography software, the scattered light signals, and other system information. An optical tomography processor includes at least one hardware processor coupled to the data storage memory and configured to execute the optical tomography software including instructions to perform spectral analysis of the scattered light signals to create a three-dimensional image map representing biomarker characteristics of the target tissue.

In further specific embodiments, the spectral analysis of the scattered light signals includes heterodyning the scattered light signals with a local oscillator light signal corresponding to frequency-shifted light from the light source. The light source may be configured for generating non-invasive light input signals to the target tissue, for generating light input signals at a plurality of different wavelengths—e.g. red and/or infrared light—and the light source may include a spatial light modulator device. The system different time-dependent waveforms may represent different ultrasound frequencies.

### BRIEF DESCRIPTION OF THE DRAWINGS

FIG. 1 illustrates the principle of conventional ultrasound-modulated optical tomography.

FIG. 2 illustrates the principle of a multi-frequency arrangement for ultrasound-modulated optical tomography.

FIG. 3 shows an arrangement for direct multi-frequency optical tomography according to an embodiment of the present invention.

FIG. 4 shows an example of acousto-optical interaction in two exemplary voxels according to an embodiment of the present invention.

FIG. 5 shows an arrangement for heterodyned multi-frequency optical tomography according to an embodiment of the present invention.

FIG. 6 shows an arrangement for heterodyned multi-frequency optical tomography using multiple wavelength input light.

FIG. 7 shows an arrangement for direct multi-frequency optical tomography using multiple wavelength input light.

FIG. 8 shows an example of the geometry for an input/sensing device according to an embodiment of the present invention.

### DETAILED DESCRIPTION

The following discussion and examples are set forth in terms of red/infrared imaging of the brain. But the various discussed techniques may be useful for any medium which is highly scattering to light. Other specific applications include other tissues (e.g. breast cancer diagnostics), imaging in turbid water, microwave probing of the brain and other tissues, microwave probing of pipes and other infrastructure, and so on. Also, the discussion is set forth using terms like “light” and “optical”, it will be understood to refer generically to electromagnetic radiation, which could be any specific frequency from ultraviolet to radio.

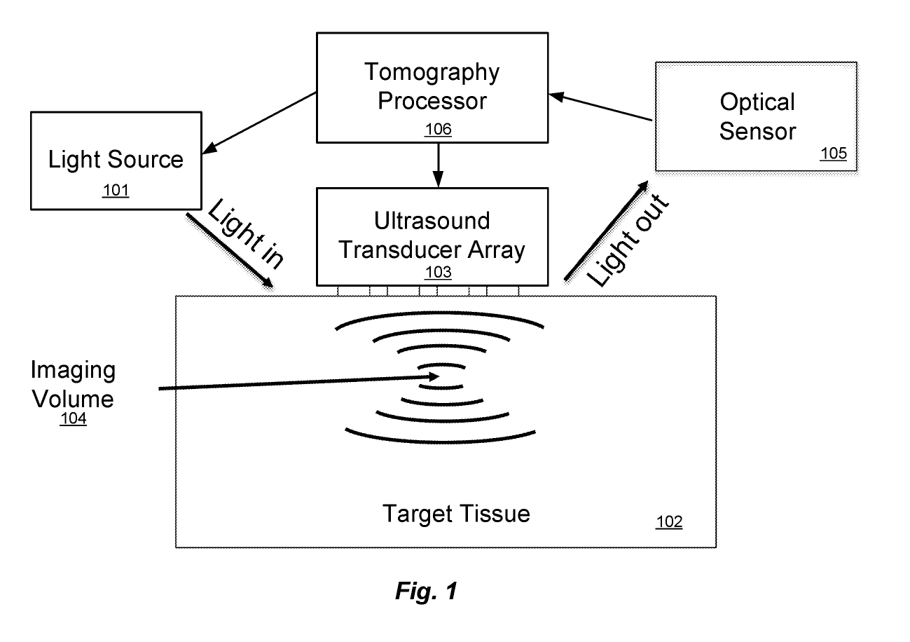

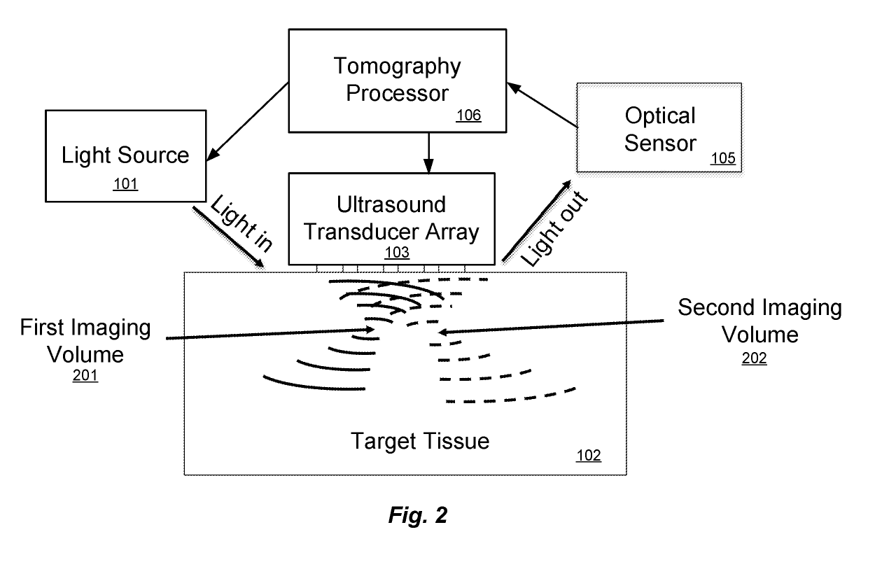

FIG. 2 illustrates the operating principle for a multi-frequency arrangement for ultra-sound modulated optical tomography, derived from the system that was discussed with respect to FIG. 1. Each transducer element of the ultrasound transducer array 106 can be considered as being attached to an arbitrary waveform generator, as an example. The tomography processor 106 can then simultaneously focus 5 MHz ultrasound into a first imaging volume 201, and 5.1 MHz ultrasound into a different second imaging volume 202, simply by superimposing the corresponding ultrasound waveform patterns from the ultrasound transducer array 103. The optical sensor 105 and the tomography processor 106 then can simultaneously monitor the 5 MHz and 5.1 MHz scattered light sidebands to simultaneously determine information from each of these imaging volumes. This approach can be extended into as many simultaneous imaging volumes as desired, at least up to the resolution limitations imposed by the ultrasound wavelength.

The multi-frequency tomography approach illustrated in FIG. 2 illustrates the general principle that, if each transducer in an array emits a different time-dependent waveform, then a spatial map can be inferred from the time-domain output signal. There are many ways to apply this general principle by choosing a set of time-dependent waveforms for the transducers; as one illustrative example, each transducer in an array could emit an ultrasound wave following a code-division multiple access (CDMA) protocol. However, it could be challenging to generate complicated waveforms for each of hundreds or thousands of ultrasound transducers. For this reason, an especially convenient implementation involves driving each transducer in an array as a pure sinusoid with a different frequency for each transducer. In other words, in the FIG. 2 approach, there is a complicated waveform for each transducer and a very simple (1-to-1) relationship between the scattered light sidebands and the imaging volumes. But that can be reversed so that there is a simple sinusoidal waveform for each ultrasound transducer, but a more complicated and indirect relationship between the sideband amplitudes and phases on the one hand, and the three-dimensional geometry of the target tissue on the other hand.

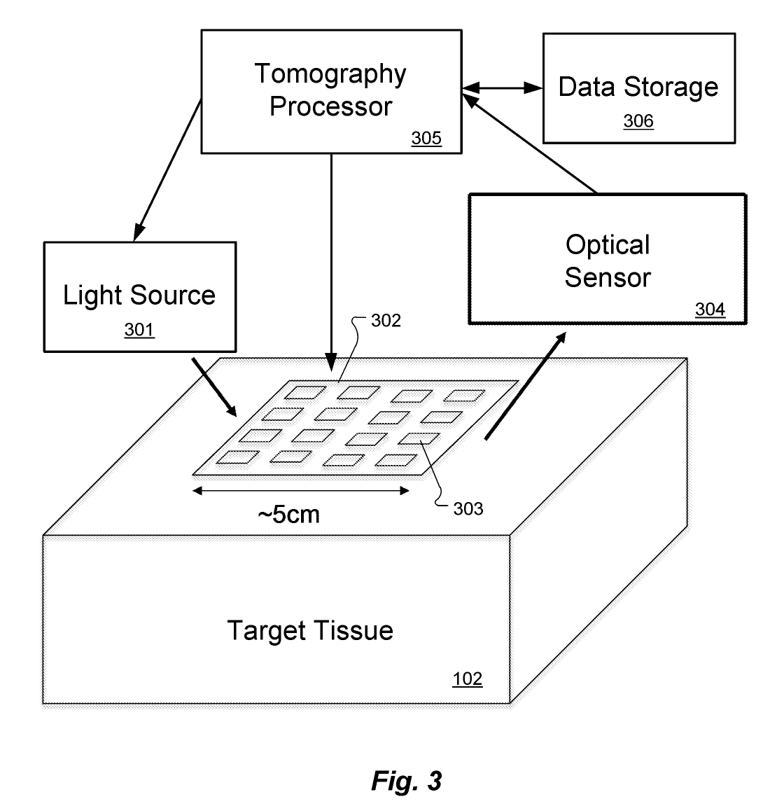

FIG. 3 shows an arrangement for direct multi-frequency ultrasonically-encoded optical tomography of target tissue such as a brain of a patient according to an embodiment of the present invention. Light source 301 (e.g. laser, superluminescent diode, LED, etc.) is configured for generating light input signals to the target tissue 102, for example, to shine light into the brain. The input light signals from the light source 301 can be sent from a single point, or from several different points, or from a larger-area (defocused) spot. The light source 301 can produce the light input signals non-invasively, if the light is in a wavelength range where the skin and skull are sufficiently transparent or translucent (e.g., red and/or near infrared).

An ultrasound transducer array 302 is configured for placement on the outer surface of the target tissue and has multiple ultrasound transducers 303 each operating at a different ultrasound frequency to generate ultrasound input signals to an imaging volume within the target tissue 102. The ultrasound transducer array 302 might specifically have, for example, 10,000 individual ultrasound transducers 303 on it arranged in a 100×100 square. There may be as few as 10 total ultrasound transducers 303, or as many as 100,000, and they could be arranged in various possible shapes such as a square, circle, annulus, several patches, etc. The spacing between the ultrasound transducers 303 may usefully be related to half the ultrasound wavelength (typically 1 mm or less). A different continuous-wave ultrasound frequency is applied to each individual ultrasound transducer 303. For example, one ultrasound transducer 303 may be vibrating at 5.0000 MHz, another might be at 5.0001 MHz, and so on. For discussion clarity, ultrasound scattering, refraction, etc. will be omitted and it is assumed that each ultrasound transducer 303 creates clean, smooth, outgoing spherical wavefronts in the target tissue 102. (The effects of ultrasound scattering, refraction, etc. are discussed further below.)

An optical sensor 304 is configured for sensing scattered light signals from the imaging volume in the target tissue 102, wherein the scattered light signals include light input signals modulated by acousto-optic interactions with the ultrasound input signals. The optical sensor 304 may specifically include a multi-mode fiber or fiber bundle that takes light scattering out of the target tissue 102 from one or more specific locations and aims it onto a fast single-pixel detector.

Data storage memory 306 is configured for storing optical tomography software, the scattered light signals, and other system information. An optical tomography processor 305 includes at least one hardware processor coupled to the data storage memory and configured to execute the optical tomography software including instructions to perform spectral analysis of the scattered light signals from the optical sensor 304 to create a three-dimensional image map representing biomarker characteristics of the target tissue 102.

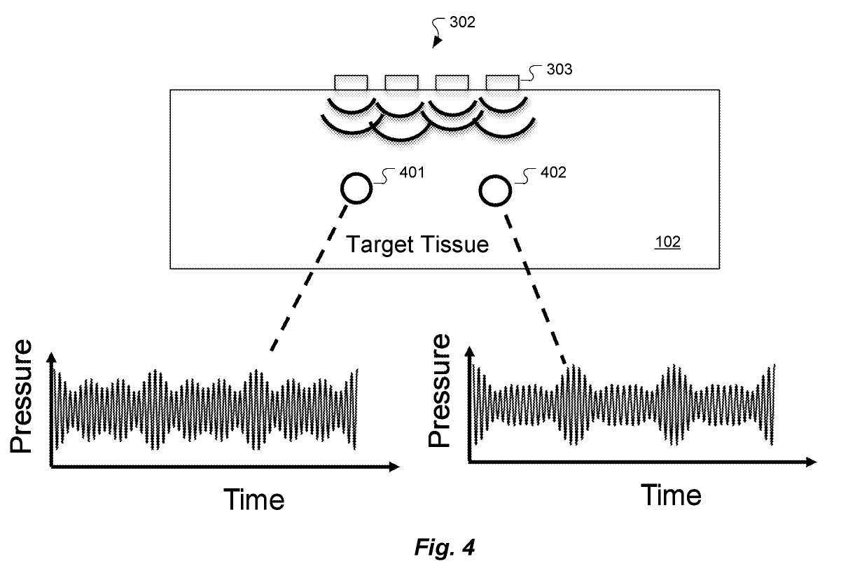

Due to the different ultrasound frequencies, each specific location in the target tissue 102 is subjected to a different time-dependent waveform, distinguished by the relative phase and amplitude of each frequency component. For example, in FIG. 4, the ultrasonic waveforms at two different imaging volumes 401 and 402 are shown (in a schematic, not literal, way). They look different primarily (though not exclusively) because they have different propagation-related phase delays to each of the ultrasound transducers 303. The scattered light in the target tissue 102 is modulated by acousto-optic interactions from the ultrasound signals. For example, a 5.4321 MHz ultrasound transducer causes the light intensity reaching the optical sensor 304 to oscillate at 5.4321 MHz. Spectral analysis of the scattered light signal should show a peak at 5.4321 MHz, and the amplitude and phase of this peak reflects the amplitude and phase with which the ultrasonic waves from this particular transducer are interacting with the light, in the aggregate.

The spectral analysis performed by the tomography processor 305 includes a post-processing step that converts the amplitude and phase information associated with each ultrasound transducer into the three-dimensional map. This can be thought of (in many ways) as a “holographic reconstruction”. The spectral analysis may be based on a computer model that treats each ultrasound transducer as emitting an ultrasound wave with the phase and amplitude inferred from the amplitude and phase of the corresponding frequency component of the detector data. (The phase may or may not need to be sign-flipped, depending on the sign conventions used.) As all these waves propagate and interfere in the computational simulation, they create a three-dimensional intensity profile corresponding to the three-dimensional map that is sought. This computer model should include effects such as ultrasound refraction, diffraction, reflection, and scattering (to the extent that these are known).

The three-dimensional map produced by the tomography processor 305 reflects the product of local light intensity, local light output probability (i.e. the probability for light at this point to eventually reach the optical sensor 304), and acousto-optic coefficient (which in turn is related to refractive index and other properties of the materials and their configuration).

With reference the simple example shown in FIG. 4, suppose that acousto-optic interaction occurs in the two indicated small imaging volumes 401 and 402 and nowhere else. Then the detector intensity as a function of time at the optical sensor 304 would appear as a weighted sum of the two waveforms shown. In the holographic reconstruction step of the data analysis, the tomography processor 305 would assign to each ultrasound transducer 303 the amplitude and phase inferred from the corresponding Fourier component of the detected scattered light intensity waveform in a computational acoustic wave propagation simulation. If the ultrasound transducers 303 were hypothetically emitting waves with these amplitudes and phases, they should add coherently to a high intensity at the two small circles of the imaging volumes 401 and 402 and to a much lower intensity everywhere else.

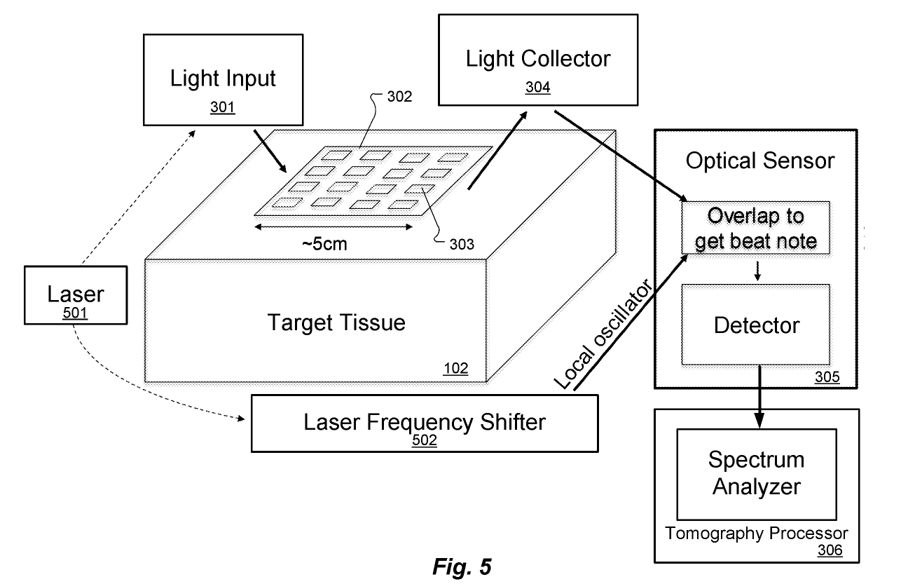

FIG. 5 shows an arrangement for heterodyned multi-frequency optical tomography according to an embodiment of the present invention, which may be a bit more complicated to implement, but may have an improved signal-to-noise ratio (SNR). Laser light from laser 501 is split into two branches (typically fibers). One of these branches is used by the light input 301 to shine light into the target tissue 102 as described above. The other branch of the laser light from laser 501 is frequency shifted by some amount “f_shift” by laser frequency shifter 502. This can be done using standard methods such as an acousto-optic modulator, electro-optic modulator, intensity modulator, frequency offset lock, frequency comb techniques, etc. The output light from the laser frequency shifter 502 represents a local oscillator signal. The optical sensor 305 includes a heterodyne light detection arrangement that processes the scatter light from the light collector 304 and the local oscillator signal from the laser frequency shifter 502. This involves overlapping the two light signals onto a fast detector which then sees amplitude modulation related to beat notes. And as above, this is processed by the spectrum analyzer of the tomography processor 306.

Due to acousto-optic interactions, if (for example) 400 THz light goes into the brain, the scattered light exiting is mostly 400 THz, but in the example above it would have sidebands at (400 THz±5.0000 MHz), (400 THz±5.0001 MHz), etc. The spectrum analyzer in the tomography processor 306 should therefore see a strong peak at frequency f_shift, with 10,000 pairs of sidebands, one pair for each ultrasound transducer 303. Each pair of sidebands is caused by one particular ultrasound transducer 303, and analysis of the detector output will yield the amplitude and phase with which the ultrasonic waves from this particular ultrasound transducer 303 are interacting with the light, in the aggregate. The post-processing analysis (“holographic reconstruction”) is as above.

In the embodiment in FIG. 5, the local oscillator is a separate light beam, while in the embodiment in FIG. 3, the function of the local oscillator is performed by the non-frequency-shifted light sensed by the optical sensor 305, i.e. the fraction of light that enters and exits the target tissue 102 without interacting with the ultrasound signals. From this consideration, it follows that the heterodyne embodiment in FIG. 5 may be likely to have a higher signal-to-noise ratio than the embodiment in FIG. 3. The explicit local oscillator signal in FIG. 5 can be much stronger because it bypasses the target issue 102 and so is not constrained by safe exposure limits. Moreover, in the embodiment in FIG. 5, various high-sensitivity heterodyne detection techniques can be used (or else used more effectively), such as intensity stabilization of the local oscillator, balanced detection, choosing an f_shift that places the sidebands at a frequency most advantageous for high-SNR detection (e.g. low noise and background and systematics), and so forth. On the other hand, the embodiment in FIG. 3 has its own advantages such as simpler hardware and better compatibility with LEDs (as opposed to lasers).

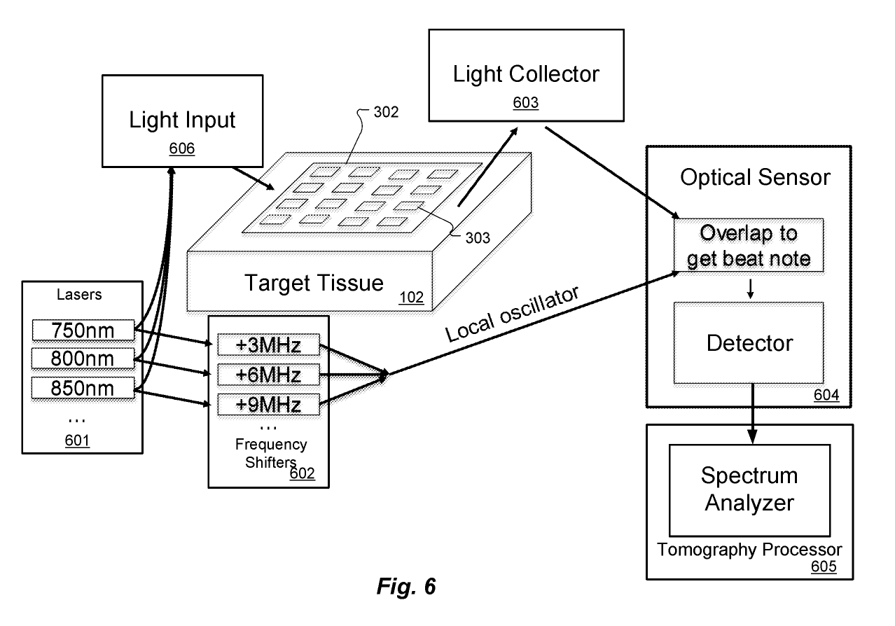

FIG. 6 shows an arrangement for heterodyned multi-frequency optical tomography using multiple wavelength input light simultaneously without sacrificing spatial or temporal resolution and without even needing more than one heterodyne detection module. Lasers 601 create laser light with several different wavelengths for light input 605. The laser light from lasers 601 also is shifted by frequency shifters 602 each by a different frequency in order to create the corresponding local oscillator signal. The light input 606 carries the light signals to the target tissue 102 (either combined or in separate fibers), while the local oscillators are combined and sent to the heterodyne unit within the optical sensor 604. The heterodyne unit sees a complete set of sidebands related to the first wavelength, and, at a different center frequency, a complete set of sidebands related to the second wavelength, and so on. With appropriate frequency choices, these sets of sidebands in the scattered light from the light collector 603 will not overlap, or may only overlap a limited extent, so that they can be separated by the tomography processor 605 in post-processing.

An equivalent functionality could also be accomplished using frequency comb techniques somewhat along the lines of dual-comb spectroscopy. More specifically, the light input would be one frequency comb, and the local oscillators would be a different comb. If the two combs have different teeth spacing, the result would be similar to that in FIG. 6.

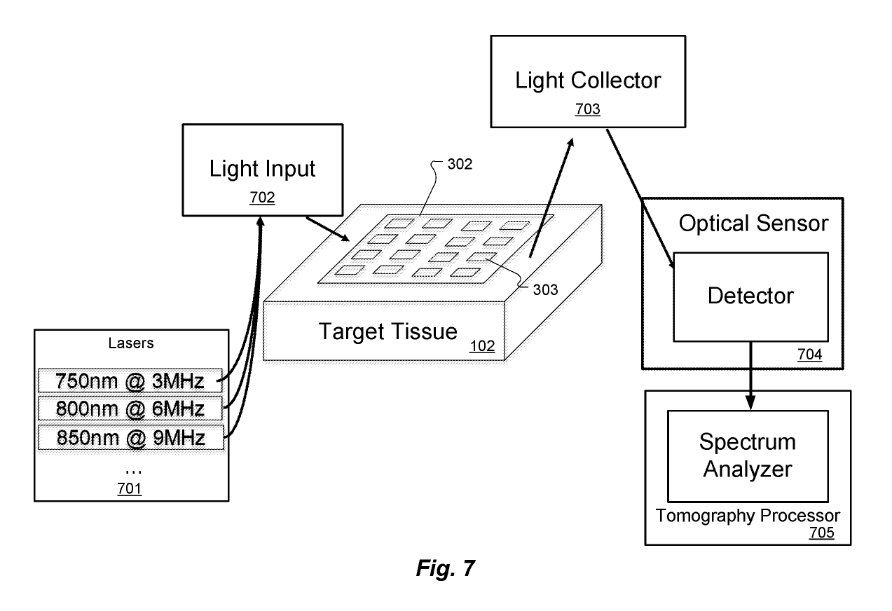

FIG. 7 shows an embodiment for direct multi-frequency optical tomography using multiple wavelength input light without explicit local oscillators or heterodyning. A bank of lasers 701 (or LEDs) is used, and each different wavelength is amplitude-modulated (most simply, switched on and off) at a different rate for delivery to the target tissue 102 by light input 702. This causes sidebands to be duplicated at higher frequencies in the scattered light from the light collector 703 to the optical sensor 704, and hence the tomography processor 705 can extract the different wavelength sidebands with a similar result as in the embodiment in FIG. 6.

One advantageous feature of such arrangements is its speed. New data points are obtained as quickly as the inverse separation between transducer frequencies (e.g. 100 Hz). Partial information is available even faster, though that is more difficult to interpret (but not impossible). And this is a whole three-dimensional image at each 1/(100 Hz) interval, not just one imaging volume (voxel) at a time, and indeed, in multiple-wavelength embodiments, it is a whole three-dimensional image with spatially-resolved spectral information.

This quasi-continuous monitoring can be advantageous for many different applications. One example is mapping brain activation patterns for purposes such as psychological studies, psychiatric diagnoses, brain-machine interfaces for paraplegics, and others. These activation patterns have important high-speed dynamics which usefully can be captured, and for brain-machine interfaces, it is critical to minimize the delay between brain activation and its detection. Another example is that with a high data rate, an embodiment can effectively perform computational correction for motion of the ultrasound transducer array relative to the imaged anatomical features. Implementation would be generally along the lines of the digital image stabilization techniques used in many cameras. Another example is that with a high data rate, a variety of temporal filters can be applied to extract additional information. For example, it is possible to extract just the image or spectral changes that are in synchrony with the pulse rate, by combining measurement data with a heart-rate monitor and then using typical lock-in amplifier-type techniques. Or conversely, the pulse-related changes can be suppressed in the data output. As another example, frequency filtering may enable the sensing of neural activity such as gamma waves.

Another appealing feature is the image resolution, which should be comparable to the ultrasound frequency used, typically 1 mm or less, which is similar to fMRI. Embodiments also provide good signal-to-noise ratio (SNR)—low-noise high-sensitivity heterodyne receivers can be implemented via various known techniques including, for example, balanced detection, local oscillators with high power and intensity stabilization feedback, etc. Embodiments can be implemented at favorably low size, weight, power, and cost. For example, the input light is single-pixel in the sense a spatial light modulator (SLM) is not required, and the output light is also single-pixel in the sense that there is no detector array required. Although the ultrasound transducers must be driven with many different frequencies, it helps that each is following a simple continuous sinusoidal waveform, which is generally easy to synthesize.

It might be useful to include a spatial light modulator (SLM) as part of the light source module, particularly in order to improve the efficiency with which light transmits into (and back out of) the general region being imaged, particularly through the skin and skull. (See “Light finds a way through the maze”, John Pendry, Physics 1, 20 (2008)). The SLM settings could be optimized using existing 3D data available through the device, as this data indirectly indicates the three-dimensional light intensity profile, conveniently including only those photons which eventually reach the optical sensor. While it would increase system complexity, this could provide higher (perhaps dramatically higher) signal-to-noise ratio if input light power is held constant, or reduced light input power for the same signal-to-noise ratio (reducing the risk of skin burning etc.). If a multi-mode fiber is used to carry the input light, the SLM could be located before the light enters the fiber, rather than at the patient's head. An SLM is not the only non-invasive way to increase light transmission through the skin and skull and into a region of interest, which could also involve finely adjusting the optrode angle, and/or position, and/or light wavelength, in order to find a configuration where transmission into the region of interest is higher than usual. Similarly, there could be a spatial light modulator or other adjuster at the output side, in order to increase the efficiency with which light, having exited from the tissue, reaches the small detector.

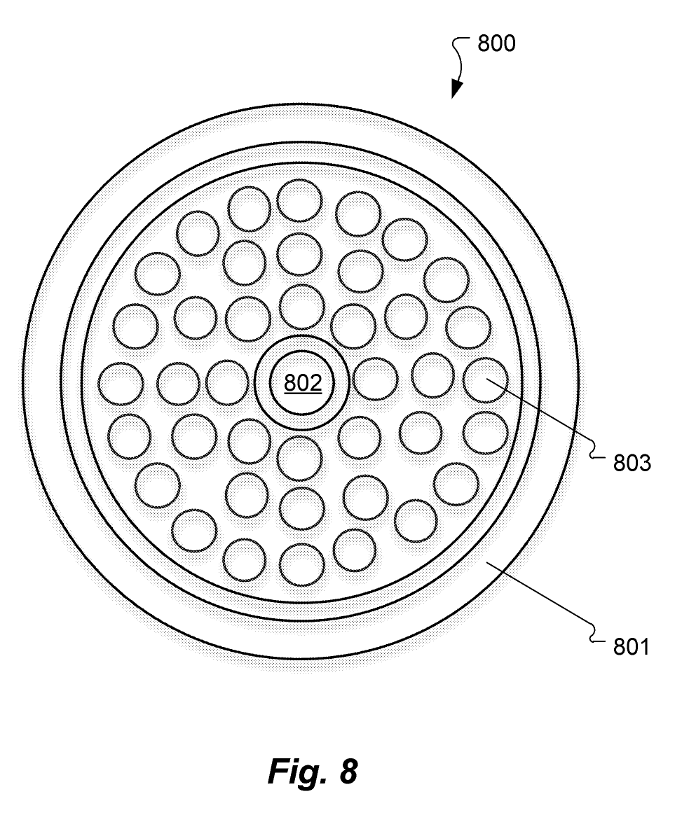

FIG. 8 shows an example of the geometry for an input/sensing device 800 according to an embodiment of the present invention which combines the ultrasound transducer array 803, light input 801, and light collector 802. The light input 801 is formed as a large ring that produces a larger volume of illumination and more uniformity. The light collector 802 extracts the modulated scattered light signals from the center of the input/sensing device 800, and ultrasound transducer array 803 fills the annular space between them and provides the acousto-optic interaction required for position resolution.

Overall, the geometrical arrangement of which transducers use which frequency does not matter much, however, this design parameter can have some indirect consequences. For example, pairs of transducers with especially close frequencies—for example 5.4792 MHz vs. 5.4793 MHz—should probably be placed farther apart from each other to reduce undesirable cross-talk via electrical and/or mechanical coupling.

The modulated scattered light output could be tapped at multiple points and/or fed into multiple heterodyne detectors to improve SNR. This might be accomplished as simply as putting multiple fast detectors side-by-side in the same optical sensor unit.

Typically an optical diode protects the laser light source. And the path lengths of the two optical paths to the heterodyne receiver should be approximately equal. The laser linewidth should be sufficiently narrow and frequency sufficiently stable so as to obtain high-contrast narrow-bandwidth beat notes that are spectrally well separated from each other. For example, a 1 GHz linewidth allows heterodyne beat notes to be visible with up to about 1 foot of optical path length discrepancy between the two paths that are being interfered. On the other hand, subject to these constraints, the laser frequency could be dithered or broadened to a certain extent to reduce the distracting effects of laser speckle in the images.

A single instrument could potentially be configured to take measurements using both the modality described above, and also other modalities such as traditional ultrasound, photoacoustic imaging, various fNIRS or diffuse optical tomography techniques, and so on. For example, a traditional ultrasound scan could reveal the acoustic scattering, speed of sound profile, and other parameters that could make the “holographic reconstruction” step (see above) more accurate. As another example, the technique here could be combined with focused ultrasound brain stimulation, in order to not only read but also modify neurological states. As still another example, the technique here could be combined with high-intensity focused ultrasound in order to destroy a tumor while monitoring progress.

Higher-order acousto-optic interactions could produce extra sidebands or contribute to already existing sidebands in the modulated scatter light, for example, at the ultrasound sum- or difference-frequencies. It may be beneficial to reduce the ultrasound amplitude sufficiently to minimize these types of interactions and so make the data analysis more tractable. However, to the extent that they are present, they could be used in the spectral analysis and could even increase the image resolution (because sum-frequency waves have a shorter wavelength).

As previously mentioned, the computational ultrasound wave propagation part of the holographic reconstruction process should account for effects such as ultrasound refraction, diffraction, reflection, and scattering, to the extent that these are known. These parameters can be predicted from typical anatomy and/or measured by conventional ultrasound and/or inferred from the three-dimensional image itself. For example, assuming that sound travels at a different speed in the skull than elsewhere, then if the skull thickness profile is estimated incorrectly, it might cause the three-dimensional map to have a warped appearance with smooth surfaces appearing wavy. Using such a map, the skull thickness profile could be corrected based on prior knowledge about the shapes of anatomical features. As another example, if a surface has an incorrectly-estimated ultrasound reflection coefficient, then a spurious mirror-reflected copy of features might appear in the three-dimensional map. But this duplication, if recognized, could be used to correct the ultrasound reflection coefficient in the computer model, thus fixing or mitigating the erroneous duplication and so improving the fidelity of the map.

Spectroscopic information can also be obtained by using optical filters to split up different wavelengths, and then having one heterodyne detector for each wavelength. This increases the system complexity but may increase SNR. Spectroscopic information also can be obtained simply by turning one wavelength on, then the next wavelength, etc. But that would impair temporal resolution and perhaps SNR.

There are two prior techniques known in the literature that are somewhat similar to what is described herein in the sense that: (1) three-dimensional spatially-resolved and potentially spectrally-resolved information is obtained, and (2) the resolution is related to ultrasound wavelengths because ultrasound is ultimately used to encode or detect the position. One such approach is known by various terms including ultrasonically-encoded optical tomography, acousto-optic tomography, or ultrasound guide star; see “Time-reversed ultrasonically encoded optical focusing into scattering media”, Xu et al., Nat. Phot. 5, 154 (2011)(incorporated herein by reference in its entirety). Another such approach is known as photoacoustic imaging; see e.g., “Imaging cancer with photoacoustic radar”, Mandelis, Physics Today 70, 42 (2017)(incorporated herein by reference in its entirety). But in their specifics, these two techniques are very different from each other and from the technique described herein.

Photoacoustic imaging uses a very different detailed mechanism, using light to create ultrasonic waves and then detecting that ultrasound with piezo transducers, whereas the embodiments of the present invention described herein use piezo transducers to create ultrasonic waves that modulate light in a way that is detected optically. So in one sense, the two different approaches are opposites. In addition, embodiments of the present invention enable a better signal-to-noise ratio, and allows measuring many wavelengths at once without losing spatial or temporal resolution. Moreover, photoacoustic imaging measures almost purely absorption, whereas embodiments of the present invention are also sensitive to acousto-optic coefficient, which is related to refractive index and other parameters. In this respect, the two different techniques might be complementary, and, as mentioned above, it is conceivable that the same system devices could support both sensing modalities.

Ultrasonically-encoded optical tomography has previously generally used single-frequency ultrasound phased arrays (as in FIG. 1), and therefore image one voxel at a time, and usually also one wavelength at a time. Thus it has been a slow technique. One variant of ultrasonically-encoded optical tomography uses a spatial light modulator (SLM) on the input light. The SLM's phase map is set to focus light of a certain wavelength onto a certain voxel (imaging volume). This phase map is computed using an ultrasound array that focuses sound waves to a particular voxel. In a dynamic living tissue, this variant can be even slower, because it is not only one-voxel and one-wavelength-at-a-time imaging, but also it requires that each of the phase maps be periodically re-measured or re-optimized due to the ever-changing microscopic scattering pattern.

Even though embodiments of the present invention have been discussed in terms of using an SLM on the input light, the purpose and details are quite different. In ultrasound guide star (and other known techniques), the SLM is used to focus light to one voxel, and then get data just about that one voxel, with a separate phase map for each voxel. In embodiments of the present invention, the SLM is provides more light into a relatively large-volume general region (e.g., through the skull into the brain and/or deeper into the brain and/or in the general direction of the light output) much larger than an image voxel. Spatial resolution comes from the ultrasound frequency encoding, not from the SLM, and hence this technique can get images much faster, and with greatly reduced requirements on the speed, size, resolution, and location of the SLM.

Diffuse optical tomography typically just sends light in at one point and collects it at another point. Hence it is far lower resolution than the approach used in embodiments of the present invention, which gets a whole three-dimensional map for each input and output rather than merely one data point. For example, “Mapping distributed brain function and networks with diffuse optical tomography”, Nature Photonics 8, 448 (2014) by Eggebrecht et al. refers to ˜1.5 cm resolution as “high-density diffuse optical tomography”, even though it probes perhaps 3 orders of magnitude larger volume elements than the approach described above for embodiments of the present invention (cm³ instead of mm³). fNIRS (functional near infrared spectroscopy) methods all have similar resolution limitations. Optical coherence tomography (OCT) has higher resolution, but much shallower depth in highly-scattering tissues, since OCT uses photons that only scatter once, whereas the present invention can get good data from photons that have scattered very many times.

Magnetic resonance imaging (MRI) senses different characteristics than light does and also has extremely high size, weight, power, and cost, and is not portable, and generally cannot be used on patients with metal implants (e.g. pacemakers, cochlear implants, etc.). Positron-emission tomography (PET) also observes different characteristics than light does, and has high size, weight, power, and cost, and is not portable, and is sometimes not usable due to the ionizing radiation. Ultrasound (by itself) similarly observes different characteristics than light does. EEG and MEG tend to have far lower resolution than the sub-mm voxels discussed here, and again, they see very different things than light does.

Embodiments of the invention may be implemented in part in any conventional computer programming language such as VHDL, SystemC, Verilog, ASM, etc. Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A computer-implemented system for multi-frequency ultrasonically-encoded optical tomography of target tissue having an outer surface, the system comprising:
a light source configured for generating light input signals to the target tissue;
an ultrasound transducer array configured for placement on the outer surface of the target tissue and having a plurality of ultrasound transducers each generating a different time-dependent waveform to form a plurality of ultrasound input signals to an imaging volume within the target tissue;
an optical sensor configured for sensing scattered light signals from the imaging volume, wherein the scattered light signals include light input signals modulated by acousto-optic interactions with the ultrasound input signals;
data storage memory configured for storing optical tomography software, the scattered light signals, and other system information;
an optical tomography processor including at least one hardware processor coupled to the data storage memory and configured to execute the optical tomography software including instructions to perform spectral analysis of the scattered light signals to create a three-dimensional image map representing biomarker characteristics of the target tissue.

2. The system according to claim 1, wherein the spectral analysis of the scattered light signals includes heterodyning the scattered light signals with a local oscillator light signal corresponding to frequency-shifted light from the light source.

3. The system according to claim 1, wherein the different time-dependent waveforms represent different ultrasound frequencies.

4. The system according to claim 1, wherein the light source is configured for generating non-invasive light input signals to the target tissue.

5. The system according to claim 1, wherein the light source is configured for generating light input signals at a plurality of different wavelengths.

6. The system according to claim 1, wherein the light source contains a spatial light modulator device.

7. The system according to claim 1, wherein the light input signals include at least one of red light and infrared light.

8. The system according to claim 1, wherein the target tissue includes a brain of a patient

9. A computer-implemented method employing at least one hardware implemented computer processor for multi-frequency ultrasonically-encoded optical tomography of target tissue having an outer surface, the method comprising:
operating the at least one hardware processor to execute program instructions for:
generating light input signals to the target tissue;
operating an ultrasound transducer array placed on the outer surface of the target tissue and having a plurality of ultrasound transducers each generating a different time-dependent waveform to form a plurality of ultrasound input signals to an imaging volume within the target tissue;
sensing scattered light signals from the imaging volume, wherein the scattered light signals include light input signals modulated by acousto-optic interactions with the ultrasound input signals; and
performing spectral analysis of the scattered light signals to create a three-dimensional image map representing biomarker characteristics of the target tissue.

10. The method according to claim 9, wherein performing spectral analysis of the scattered light signals includes heterodyning the scattered light signals with a local oscillator light signal corresponding to frequency-shifted light from the light source.

11. The method according to claim 9, wherein the different time-dependent waveforms represent different ultrasound frequencies.

12. The method according to claim 9, wherein the light input signals are non-invasive light input signals.

13. The method according to claim 9, wherein the light input signals have a plurality of different wavelengths.

14. The method according to claim 9, wherein the light input signals are modulated by a spatial light modulator device.

15. The method according to claim 9, wherein the light input signals include at least one of red light and infrared light.

16. The method according to claim 9, wherein the target tissue includes a brain of a patient
